(19) United States
(12) Patent Application Publication
(10) Pub. No.: US 2024/0193779 A1
(43) Pub. Date: Jun. 13, 2024

(54) SYSTEM FOR HELPING DIAGNOSING AGGRESSIVE PROSTATE CANCERS AND RELATED METHOD

(71) Applicants: INSTITUT NATIONAL DE LA SANTE ET DE LA RECHERCHE MEDICALE, Paris (FR); UNIVERSITE GRENOBLE ALPES, Saint Martin D'Heres (FR); CENTRE NATIONAL DE LA RECHERCHE SCIENTIFIQUE, Paris (FR); HOSPICES CIVILS DE LYON, Lyon (FR); CENTRE LEON BERARD, Lyone (FR); UNIVERSITÉ CLAUDE BERNARD LYON 1, Villeurbanne (FR)

(72) Inventors: Rémi SOUCHON, Lyon (FR); Tristan JAOUEN, Lyon Cedex 03 (FR); Au HOANG-DINH, Hanoi (VN); Christelle GONINDARD, Grenoble Cedex 9 (FR); Olivier ROUVIERE, Lyon (FR)

(21) Appl. No.: 18/555,618

(22) PCT Filed: Apr. 27, 2022
(86) PCT No.: PCT/EP2022/061221

§ 371 Date: Oct. 16, 2023

(30) Foreign Application Priority Data
Apr. 28, 2021 (EP) 21305545.2

(51) Int. Cl. G06T 7/00

(52) U.S. Cl. CPC G06T 7/0016; G06T 2207/10088; G06T 2207/30081

(57) ABSTRACT

The invention relates to a system and a method for computer-aided detection (CAD) of aggressive prostate cancers. The automatic diagnosis of aggressive prostate cancers is difficult and methods that work with some MRI scanners do not work as well when implemented on images from different scanners. By studying a data base containing MRI data and biopsy results from 265 patients, acquired by 4 different types of MRI scanners, using machine learning techniques, the inventors established a method for automatically determining the presence of aggressive cancers, this methods showing high sensitivity and specificity when implemented on another database containing MRI data from 270 patients, acquired by different MRI scanners than the first database. The method involves feeding MRI images to a system calculating a score for a portion of the prostate and determines that the portion contains an aggressive cancer based on whether a criterion depending on the score is verified.

### FIELD OF THE INVENTION

The present invention concerns a system for helping diagnosing aggressive prostate cancers, as well as a method for helping diagnosing aggressive prostate cancers.

### BACKGROUND OF THE INVENTION

With more than one million new cases each year, prostate cancer is the most prevalent cancer in men worldwide, and the second cancer-related cause of death. The diagnosis relies on ultrasound-guided biopsies. However often ultrasound does not show the tumor, and the role of ultrasound is limited to guiding the biopsy needle into the prostate.

The practitioner typically performs 12 systematic biopsies. It is not uncommon that biopsies miss clinically significant tumors. As a consequence, the patient does not receive appropriate treatment.

In other patients, the biopsy needle may accidentally fall within an isolated spot of cancerous cells, when the actual tumor is small and indolent and is not threatening the patient's health. As a consequence, these patients receive aggressive treatment, possibly with severe adverse effects such as incontinence and impotence, with no medical benefit.

In order to improve biopsy targeting, some image processing methods that try to recognize the signs of aggressive cancer in MRI images have been proposed, for example in the articles by Au Hoang Dinh et al. “Quantitative analysis of prostate multiparametric MR images for detection of aggressive prostate cancer in the peripheral zone: a multiple imager study”, published in 2016 in the journal Radiology, volume 280, number 1, page 117, and “Characterization of prostate cancer with Gleason score of at least 7 by using quantitative multiparametric MR imaging: validation of a computer-aided diagnosis system in patients referred for prostate biopsy”, published in 2018 in the journal Radiology, volume 287, number 2, page 525.

However, the known methods remain less reliable for detecting aggressive cancers than an experienced radiologist would be. In addition, these pre-existing methods have usually been developed using a base of MR images acquired by a limited number of MR scanners, and their reliability decreases significantly when used to detect cancers on MR images acquired using a different type of MR scanner than those used in the original base.

There is therefore a need for a system, able to help a radiologist diagnose aggressive prostate cancers, that reliably detects aggressive prostate cancers while being more robust than the pre-existing systems, and notably able to be used reliably with MR images that were acquired using different types of MR scanners.

### SUMMARY OF THE INVENTION

In this view, the present specification concerns a system for helping diagnosing aggressive prostate cancers according to claim 1.

According to specific embodiments, the system is according to any one of claims 2 to 11.

The present specification also concerns a method for helping diagnosing aggressive prostate cancers according to claim 12.

The present specification also concerns a computer program product according to claim 13.

The present specification also concerns an information medium according to claim 14.

The present specification further concerns a method for diagnosing aggressive prostate cancers, comprising a step for implementing a method according to claim 12.

### BRIEF DESCRIPTION OF THE DRAWINGS

Features and advantages of the invention will appear upon reading the following specification, given only as a non-limiting example, and made with reference to the associated drawings, in which:

FIG. 1 is a schematics of a system for helping diagnose aggressive prostate cancers, and

FIG. 2 is a flow chart of the steps of an example of a method for helping diagnose aggressive prostate cancers, implemented by the system of FIG. 1,

FIG. 3 is a graph of the specificity of an example of system according to FIG. 1, as a function of several parameters,

FIG. 4 is a graph of the specificity of another example of system according to FIG. 1, as a function of several parameters, and

FIG. 5 is a graph of the specificity of yet another example of system according to FIG. 1, as a function of several parameters.

### DETAILED DESCRIPTION OF EMBODIMENTS

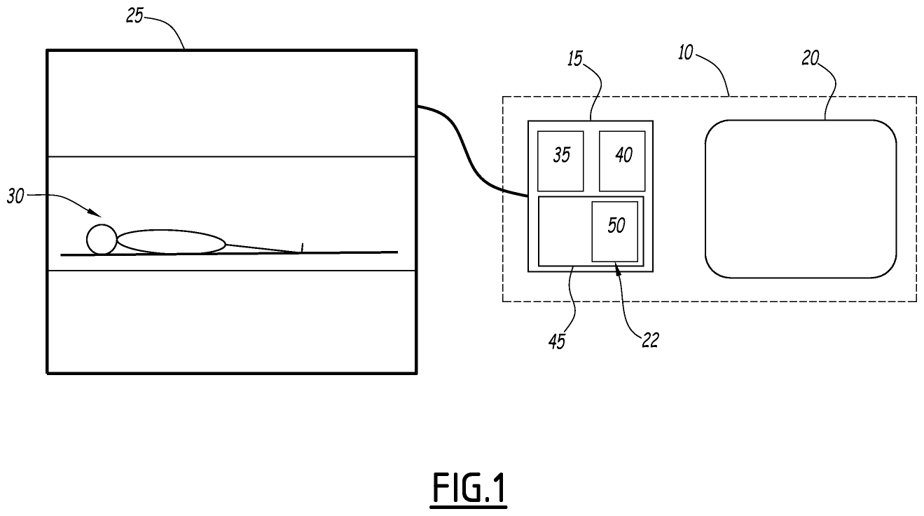

A schematics of a system 10 for helping diagnose aggressive prostate cancers is shown on FIG. 1.

The system 10 comprises a control module 15, a human-machine interface 20 and a computer program product 22.

More generally, the system 10 is an electronic computer capable of handling and/or transforming data illustrated as electronic or physical amounts in registers of the system 10 and/or memories in other similar data corresponding to physical data in the memories, registers or other types of display, transmission or memory storage devices.

The system 10 is configured to implement a method for helping aggressive prostate cancer diagnosis.

The control module 15 is configured to receive, from a distinct device 25 such as a magnetic resonance imagery scanner 25, at least one magnetic resonance image of a subject 30's prostate.

The control module 15 comprises, for example, a data processing unit, such as a processor 35, a memory 40 and a reader 45 of a legible information medium 50.

The computer program product 22 includes the information medium 50.

A legible information medium 50 is a medium legible by the data processing unit 35. The legible information medium 50 is a medium suitable for storing in memory electronic instructions and capable of being coupled with a bus of a computer system.

As an example, the legible information medium 50 is an optical disc, a CD-ROM, a magneto-optical disc, a ROM memory, a RAM memory, an EPROM memory, an EEPROM memory, a magnetic card or an optical card.

On the legible information medium 50, a program comprising program instructions is stored in memory.

The computer program may be loaded on the data processing unit 35 and is adapted for causing the application of a method for helping diagnose prostate aggressive cancers when the computer program is applied on the processor 35.

The human-machine interface 20 is configured to enable transmission of information between a human, such as a physician operating the system 10, and the control module 15.

The human-machine interface 20 comprises a display screen, for example a touch screen. Optionally, the human-machine interface 20 further comprises a mouse and/or a keyboard and/or a loudspeaker and/or a microphone.

The scanner 25 is, for example, distinct from the system 10, in particular disposed in a different room or a different building, and linked to the system 10 by a wire or wireless network.

In a possible variant, the scanner 25 is part of the system 10. For example, the control module is able to command the scanner 25 to acquire the magnetic resonance image(s).

In another variant, the distinct device 25 is a server or a database in which the magnetic resonance image(s) are stored, the magnetic resonance image(s) being in this case, for example, acquired by one or several scanner(s) and transmitted to the distinct device 25 through the network.

The scanner is a multiparametric magnetic resonance imaging (“MRI”) scanner, able to acquire images of a subject's organ using different MRI methods, as will appear below, notably by measuring the T1-weighed and T2-weighed MRI signals emitted by the organ.

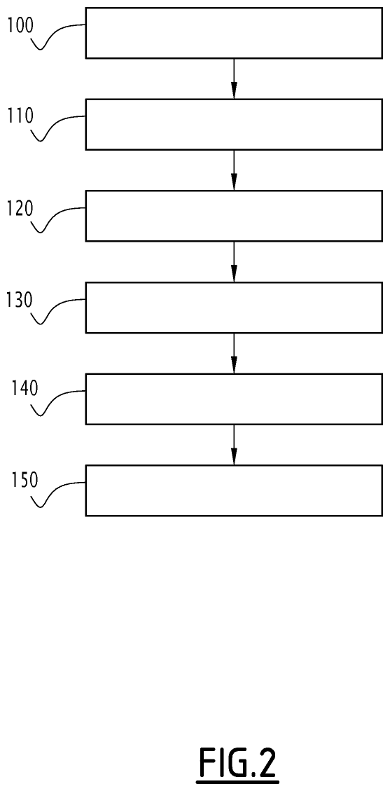

The operation of the system 10 in interaction with the computer program product is now described with reference to FIG. 2, which illustrates an exemplary application of a method for helping diagnose aggressive prostate cancers.

The method for helping diagnose aggressive prostate cancers comprises an acquisition step 100, a transmission step 110, a selection step 120, a calculation step 130, a determination step 140, and a signalling step 150.

During the acquisition step 100, at least one magnetic resonance image is acquired by the scanner 25, for example a set of magnetic resonance images.

Each image is an image of a subject's prostate.

The subject is, for example, a patient showing symptoms that are likely to be caused by a prostate cancer. In a variant, the subject does not show any symptom.

Each image is an image of at least part of the peripheral zone (“PZ”) of the subject's prostate. In particular, each image is an image of at least part of the PZ and of at least part of the transition zone (“TZ”) of the prostate.

The set of magnetic resonance image(s) comprises at least one first image and optionally one second image or several second image(s).

Each image comprises a set of picture elements or “pixels”.

The or each first image is, for example, an apparent diffusion coefficient mapping. Such a mapping is a two-dimension representation of the values of the apparent diffusion coefficient of the imaged areas of the prostate. In such a mapping, each pixel comprises a value of the apparent diffusion coefficient of a corresponding area of the prostate.

The apparent diffusion coefficient is a measure of the magnitude of diffusion of water molecules within tissue, and is obtained through diffusion-weighted imaging (DWI). Apparent diffusion coefficient values are calculated, in a per se known manner, from an initial T2*-weighed image of the prostate and a set of at least two diffusion-weighed images, each diffusion-weighed image corresponding to a spatial direction and being a mapping of the T2*-signal, attenuated as a function of how easily water can diffuse in the corresponding direction.

The apparent diffusion coefficient is expressed square millimetre per second (mm²/s).

At least one second image is, for example, a T1-weighed image comprising a set of pixels, each pixel being associated with a value of an intensity of a T1-weighed signal originating from the corresponding area of the prostate. In particular, a set of successive T1-weighed images are acquired during a time period.

The time period is a time period during which a contrast agent enters or leaves the prostate. In other words, the volumic concentration of the contrast agent increases monotonically or decreases monotonically during the time period.

For example, the contrast agent is injected to the subject, and the concentration of contrast agent increases, in the prostate and notably in each first or second area, until attaining a maximum value, and later decreases as the contrast agent is evacuated from the prostate. The T1-weighed signal intensity of each area of the prostate is a function of the contrast agent concentration, and notably increases as a function of the contrast agent concentration, although this increase is non linear. The T1-weighed signal intensity thus follows an increasing then decreasing trend, like the contrast agent concentration, over time.

The images are acquired during a time period following the injection, during which period the contrast agent enters the prostate. In a variant, the images are acquired during a time period following a moment of maximum concentration of contrast agent in the prostate, during which period the contrast agent is evacuated from the prostate after reaching its maximum concentration.

The contrast agent is, for example, gadolinium.

During the transmission step 110, all first or second images are transmitted to the control module 15.

For example, all the first or second images are acquired by the scanner 25 and immediately transmitted to the control module 15 through the network.

In a possible variant, the first or second images are sent, by the scanner 25, to a database or a server, are stored on the database or the server for some time, and are transmitted to the control module 15 after the storage period has expired, for example when a physician decides to ascertain whether or not the subject-prostate contains an aggressive cancer. In this case, the method for helping diagnose aggressive prostate cancers may be considered to not comprise the acquisition step 100.

During the selection step 120, at least one first portion of the PZ is selected.

For example, at least one image, such as the or one of the first image(s) is presented to a physician on the human-machine interface 20, and the physician delineates a part of the first image that the physician considers to be of interest, notably a part of the PZ that the physician considers to possibly contain an aggressive cancer in view of the image.

Optionally, a set of different first portions of the PZ are selected, for example by the physician delineating a plurality of areas of the first image.

In a possible variant, one image such as the or one of the first image(s) is automatically divided in a plurality of portions by the control module 15, for example by superimposing a square grid onto the image, and the control module 15 then considers each of the areas of the PZ that are delimited from each other by the grid to be one such first portion.

Optionally, at least one second portion of the prostate is selected. Each second portion is a portion of the TZ.

Each second portion is, for example, delineated by a physician or selected automatically in the same way as the first portion(s).

During the calculation step 130, the control module 15 calculates a first score P based on at least the first image(s) received. Optionally, as will be detailed below, the control module further calculates a second score Y from at least the first image(s) received.

In particular, a first score P is calculated for each first portion selected, and/or a second score Y is calculated for each second portion selected.

Each first score P is a function of at least one of a first quantity x₁, a second quantity x₂ and a third quantity x₃.

The first quantity x₁ is calculated, for the first portion considered, according to the equation:

x₁=ADC+a₁×W, (equation 1)
wherein:
a₁ is a first constant,
ADC is a percentile of the apparent diffusion coefficient values of the first portion, expressed in mm²/s, and
W is a value of a normalized wash-in rate or a value of a normalized wash-out rate of the first portion, expressed in Hertz (1 Hz=1 s⁻¹).

W is estimated from the second images.

The parameter W is for example calculated by the control module 15 by:
Calculating a wash-in or wash-out rate for each area of the first portion, each area corresponding to a same pixel in successive second images, and
Calculating W as a means, notably an arithmetic means, of the wash-in or wash-out rates of the pixels corresponding to the first portion.

The normalized wash-in rate of each area is calculated by, in a known manner, generating, from the intensity values of the pixels corresponding to the area in successive second images, a curve of the intensity of the area's a T1-weighed signal as a function of time, and estimating the slope of the curve.

The normalized wash-in or wash-out rate is, notably, estimated from a normalized T1-weighted signal, where the intensity of the T1-weighed signal of a pixel at a given time is divided by the intensity of the T1-weighed signal of the same pixel before the contrast agent enters the prostate, for example before injection of the contrast agent or before the contrast agent has reached the prostate (i.e before the start of the intensity increase).

It should be noted that different methods of normalization may be used, and are equivalent to each other since the corresponding values of parameter a₁ and of the wash-in or cash-our rates corresponding to one method of normalization can be deduced from the values corresponding to another normalization.

In a possible variant, rather than dividing all values of the T1-weighted signal by the value of this signal before injection of the contrast agent, the wash-in or wash-out rate is calculated from the slope as expressed in % of the original value.

In such a variant, a₁ could be expressed in mm²/percent rather that in mm², and the values of a₁ shown 4 would therefore be 100 times smaller than if they were expressed in mm².

It should be considered that all types of normalization of the wash-in or wash-out rates may be used indifferently when implementing the present invention, with the corresponding adaptations to the values of a₁ and of the unit used to express the wash-in or wash-out rate.

A slope, notably the maximum slope, of the increasing portion of the curve (corresponding to the progressive increase of contrast agent concentration in the area considered) is the area's normalized wash-in rate. In a possible variant, the area's normalized wash-in rate is the average slope of the increasing portion of the curve.

The wash-in or wash-out rate is called “normalized” wash-in or “normalized” wash-out when the intensity curve is made of normalized intensity values, i.e of values of the intensity of the T1-weighed signal of one pixel, or of an area of the second images, at different times, divided by an intensity of the T1-weighed signal of the same pixel or area before the contrast agent has been injected. Such a normalization enables easy comparison between wash-in or wash-out rates obtained from different MRI machines.

It should be noted that, as mentioned below, other methods of normalization can be used.

A slope, such as an average slope or a maximum slope, of the decreasing portion of the curve (corresponding to the progressive decrease of contrast agent concentration in the area considered after reaching a maximum) is the area's normalized wash-out rate. In practice, the normalized wash-out rate is often the average slope.

If the parameter W is a normalized wash-in rate, the parameter W is, for example, the arithmetic means of the normalized wash-in rates of the areas corresponding to each pixel of the first portion.

If the parameter W is a normalized wash-out rate, the parameter W is, for example, the arithmetic means of the normalized wash-out rates of the areas corresponding to each pixel of the first portion.

In a variant, the normalized wash-in or normalized wash-out rate is calculated by calculating, for each first portion of each second image, a mean value of the T1-weighed signals of all pixels within the first portion and by generating a normalized curve of the means values of the first portion calculated as a function of time. The generated time curve has the same overall shape as those of each pixel, since the means contrast agent concentration in the first portion considered increases and then decreases over time. In this case, the wash-in rate value is a slope, notably a maximum or average slope, of the increasing part of the generated time curve, and the normalized wash-out rate is a slope, for example a maximum slope or an average slope, of the decreasing part of the curve.

It should be noted that normalized wash-in or wash-out rates may be calculated for the second portion(s) in a similar manner as described above.

The parameter ADC is calculated by the control module 15 by arranging in increasing order the apparent diffusion coefficient values of the pixels corresponding to the first portion considered, the parameter ADC being a percentile of the apparent diffusion coefficient values of the first portion.

The word “percentile” means that the parameter ADC is the value of the apparent diffusion coefficient values that appears at a predetermined place in the set of arranged apparent diffusion coefficient values.

For example, the tenth percentile of the apparent diffusion coefficient values is the value of apparent diffusion coefficient that is such that ninety percents of the apparent diffusion coefficient values are strictly superior to the tenth percentile and ten percents of the diffusion coefficient values are inferior or equal to the tenth percentile.

The second percentile is the value such that ninety-eight percents of the apparent diffusion coefficient values are strictly superior to the second percentile and two percents of the diffusion coefficient values are inferior or equal to the second percentile.

It should be noted that if the number of pixels corresponding to the portion considered is not divisible by 100, the percentile can be calculated by interpolation of the two pixels between which the percentile is comprised.

Unless indicated otherwise, a value “comprised between” two limit values corresponds to a range that includes both limit values.

ADC values are often expressed in square millimeter per second (mm²/s).

When W is a normalized wash-in rate, the first constant a₁ is, for example, comprised between −6×10⁻³ mm² and −10⁻³ mm². In some embodiments, first constant a₁ is comprised between −5×10⁻³ mm² and −1.5×10⁻³ mm², for example between −5×10⁻³ mm² and −3×10⁻³ mm².

In the present specification, the sign “x” between two numbers indicates multiplication. Parameter ADC is, notably, a percentile being inferior or equal to 40 when W is a normalized wash-in rate value, in particular a percentile inferior to 30, in particular a percentile inferior to 10.

When W is a normalized wash-out rate, the first constant a₁ is, for example, comprised between 2×10⁻² mm² and 15×10⁻² mm².

Parameter ADC is, for example, a percentile being inferior or equal to 15 when W is a normalized wash-out rate value.

The second quantity x₂ is calculated, for the first portion considered, according to the equation:

x₂=ADC+b₁×TTP (Equation 2)

wherein:
b₁ is a second constant,
ADC is a percentile, comprised for example between 20 and 45, of the apparent diffusion coefficient values of all pixels corresponding to the first portion, and
TTP is a time-to-peak value of the first portion.

The second constant b₁ is, for example, comprised between 10⁻⁷ and 14×10⁻⁷ mm²/square second (mm²/s²). In some cases, the second constant b₁ is comprised between 10⁻⁷ and 9×10⁻⁷ mm²/s².

The time-to-peak value is a time, on a curve of the values of a T1-weighed signal as a function of time, between a first instant and a second instant. The first instant is a time at which, following injection of the contrast agent to the subject, the values of the T1-weighed signal start to increase (i.e an instant at which the contrast agent injected begins entering the first portion considered). The second instant is a time of maximum concentration of contrast agent in the first portion, corresponding to the maximum intensity of the T1-weighed signal.

The time-to-peak value is, for example, obtained by calculating, for each second image, a mean value of the T1-weighed signals of all pixels, by generating a curve of the means values calculated as a function of time and by measuring the time-to-peak value on the curve.

In a variant, the time-to-peak value is a means value of time-to-peak values calculated for each pixel corresponding to the first portion.

The third quantity x₃ is calculated, for the corresponding first portion, according to the equation:

x₃=c₀+c₁×ADC (Equation 3)

wherein:
c₀ is a third constant,
c₁ is a fourth constant, and
ADC is the percentile inferior or equal to 25 of the apparent diffusion coefficient values of all pixels corresponding to the first portion, for example the tenth percentile or the second percentile.

The third constant c₀ is, for example, comprised between 2.41 and 6.44, notably between 3.5 and 5.0. In particular, the third constant c₀ is equal to 4.2.

The fourth constant c₁ is comprised between −7570 and −4020 s/mm², notably between −7150 and −5270 seconds/mm².

The first score P of each first portion is calculated from at least one of the first quantity x₁, the second quantity x₂ and third quantity x₃.

The first score P is, for example, equal to one of the first quantity x₁, the second quantity x₂ and third quantity x₃.

The first score P is, in a possible variant, calculated using a logistic regression.

In statistics, the logistic regression model is used to model the probability of an event existing, for example to model the probability that the first portion contains an aggressive cancer.

The first score P is, for example, calculated using a logistic regression according to the equation:

$$z = {e}_{0} + {e}_{1} x \qquad Equation 4$$

$$P = \frac{{e}^{z}}{1 + {e}^{z}} \qquad Equation 5$$

wherein x is one of the first quantity x₁, the second quantity x₂ and third quantity x₃, e₀ and e₁ each being a constant.

In this case, the score P is a probability that the first portion considered contains an aggressive cancer.

For example, in our data, the score

$$P = \frac{{e}^{z}}{1 + {e}^{z}}$$

with x=x₁, a₁=−2.39×10⁻³, ADC being ADC2 (i.e. second centile of ADC), W being a normalized wash-in rate, e₀=3.51, e1=−5.89.103 was found to be the best logistic regression model for the presence of an ISUP≥2 cancer in the peripheral zone of the prostate.

In another variant, the first score P is obtained by calculating two or three probabilities, each probability being calculated using a logistic regression according to equation 4 for a respective quantity among the first quantity x₁, the second quantity x₂ and the third quantity x₃, and by summing the three calculated probabilities.

It should be noted that, in general, many types of scores P can be envisioned, provided that there is a bijective relationship between the score P calculated and either one of the first quantity x₁, the second quantity x₂ and third quantity x₃ or a combination (such as a sum or a weighted sum) of these quantities.

For example, the score P is obtained by dividing or multiplying one of the first quantity x₁, the second quantity x₂ and third quantity x₃ by a constant, possibly resulting in a dimensionless quantity x, the dimensionless quantity being optionally fed into equation 5 to calculate the score P.

As mentioned previously, the units used to express the parameters ADC, WI, WO and TTP that participate in the calculation of the first quantity x₁, the second quantity x₂ and third quantity x₃ may also be changed, and the parameters a and, b₁, adapted accordingly.

Optionally, during the calculation step 130, the control module 15 further calculates a second score Y for the second portion, or each second portion, of the TZ.

The second score Y is calculated as a function of a fourth quantity y. The fourth quantity y is dimensionless.

The fourth quantity y is calculated as a function of the equation:

y=d₀+d₁×ADC (Equation 6)

wherein d₀ is a fifth constant, d₁ is a sixth constant and ADC is a percentile inferior or equal to 25, notably the tenth percentile, of the apparent diffusion coefficient values in the second portion considered.

The fifth constant d₀ is comprised between 2.25 and 13.48, in particular equal to 7.9.

The sixth constant d₁ is comprised between −23820 and −4490 s/mm², notably between −15030 and −6910. In particular, the sixth constant d₁ is equal to −10740.

The second score Y is, for example, calculated using the equation:

$$Y = \frac{{e}^{y}}{1 + {e}^{y}} \qquad ( Equation 7 )$$

wherein y is the fourth quantity.

However, as is the case for the first score P, many different methods may be used to calculated the second score Y from the fourth quantity y. For example, the second score Y is equal to the fourth quantity y.

During the determination step 140, the control module 15 determines, for each first portion, whether the first portion of the PZ considered comprises an aggressive cancer or not.

In particular, the control module 15 determines whether a first criterion based on the first score P is verified, and determines that the first portion comprises an aggressive cancer if the criterion is verified.

According to an embodiment, the criterion is the fact that the first score P is inferior or equal to a first threshold, in which case the control module compares the first score P to the first threshold, and determines that the first portion comprises an aggressive cancer if the first score P is inferior or equal to the first threshold. If the first score P is strictly superior to the first threshold, the control module 15 determines that the first portion does not comprise an aggressive cancer.

This is notably the case when the first score P is equal to one of the first quantity x₁, the second quantity x₂ and third quantity x₃. In this case, the first threshold is for example called “score threshold”.

The score threshold is, for example, comprised between −0.8×10⁻³ mm²/s and 2.3×10⁻³ mm²/s when the first score P is equal to the first quantity x₁, W being a normalized wash-in rate. In particular, the score threshold is comprised between 0.55×10⁻³ mm²/s and 0.8×10⁻³ mm²/s.

When W is a normalized wash-out rate, the score threshold is, for example, comprised between −1.1×10⁻³ and 3.2×10⁻³ mm²/s. In particular, the score threshold is comprised between 0.85×10⁻³ mm²/s and 1.1×10⁻³ mm²/s.

If the first score P is equal to the second quantity x₂, the score threshold is, for example, comprised between −1.1×10⁻³ and 3.3×10⁻³ mm²/s, notably between 0.9×10⁻³ mm²/s and 1.1×10⁻³ mm²/s.

The score threshold is, for example, equal to 0.40 when the score P is equal to the third quantity x₃.

It should be noted that other criteria may be considered.

If the first score P is calculated using equations 4 and 5, the criterion is the fact that the first score P is superior or equal to a first threshold, in which case the control module compares the first score P to the first threshold, and determines that the first portion comprises an aggressive cancer if the first score P is superior or equal to the first threshold. If the first score P is strictly inferior to the first threshold, the control module 15 determines that the first portion does not comprise an aggressive cancer. The first threshold is then, for example, called “probability threshold”.

During the determination step 140, the control module 15 further determines, for each second portion, whether the second portion of the TZ considered comprises an aggressive cancer or not.

In particular, the control module 15 checks whether a second criterion is verified, and determines, for each second portion, that the second portion of the TZ considered comprises an aggressive cancer if the criterion is verified. If the criterion is not verified, the control module 15 determines that the second portion does not comprise an aggressive cancer.

According to an embodiment, the control module compares the second score Y to a second threshold, and determines that the second portion comprises an aggressive cancer if the second score Y is inferior or equal to the second threshold. In this case, the criterion is the fact that the second score Y is inferior or equal to the second threshold. However, other criteria may be considered.

For example, the control module 15 calculates a probability that the second portion comprises an aggressive cancer, notably using equation 6, and determines that the second portion comprises an aggressive cancer if the probability is superior or equal to a second probability threshold.

The second probability threshold is, for example, comprised between 0.70 and 0.77 when the value ADC used in equation 6 is a twenty-fifth percentile of ADC.

If the control module 15 has determined that one portion among the first and second portion(s) comprises an aggressive cancer, the control module 15 generates, during the signalling step 150, a message directed to a physician.

The message is intended to be transmitted to the physician by the human-machine interface 20.

The message is a message informing the physician that at least one first or second portion has been determined to contain an aggressive cancer.

The message comprises, for example, an indication of the first or second portion(s) containing an aggressive cancer or aggressive cancers, such as one of the first or second images onto which the first or second portion(s) containing an aggressive cancer or aggressive cancers is (are) delineated in red, or is (are) accompanied by a written message stating that the first or second portion considered is highly likely to be a cancer.

The message may, optionally, be accompanied by a sound indicating the discovery of a cancer, such as an alarm signal.

It should be noted that the message may take any form allowing the information to be understood by the physician.

The physician can then observe in details, on the first or second image(s) or using other methods the first or second portion(s) indicated in the message, in order to determine whether the physician shares the control module's assessment regarding the presence of an aggressive cancer in the first or second portion(s).

In addition, the method enables a physician who has studied the first and second images to have his analysis of the subject's prostate counter-checked by the system 10, which makes it possible to diagnose cancers that have been missed by the physician if the system directs the physician's attention to the possible presence of a cancer in an area of the prostate that may have been overlooked by the physician or in which the cancer does not appear clearly.

In a possible variant, the signalling step 150 is not implemented, and the fact that the control module 15 has determined the presence of an aggressive cancer is, for example, simply stored in the memory 40.

The method for helping diagnose aggressive prostate cancers is, for example, implemented as part of a method for diagnosing prostate cancers, comprising a diagnosis step wherein a prostate cancer is diagnosed as a function of the message(s) generated during the method for helping diagnose aggressive prostate cancers.

In particular, the method for diagnosing prostate cancers is part of a method for treating a prostate cancer, comprising a step for treating a prostate cancer diagnosed by implementing of the method for diagnosing prostate cancers.

The equations 1 to 3 and 5 have been determined by the inventors during the course of a study using a learning database containing MRI sequences from 265 patients who had undergone successively biopsies, multiparametric MRI and a radical prostatectomy because they have been diagnosed and treated for aggressive prostate cancer.

T2-weighted, diffusion-weighted and dynamic contrast-enhanced imaging were systematically recorded and prospectively reviewed by two independent radiologists. On each sequence, they delimited regions suspected to be cancerous. A computer program extracted twenty-three quantitative parameters from these regions.

The learning database contained MR images acquired by several different MR scanners of different manufacturers and types, using different values of magnetic field:
A Siemens Symphony scanner (1.5 Tesla, 63 patients, 233 suspicious lesions),
A General Electric Medical Systems Discovery MR 750 scanner (3 T, 124 patients, 272 lesions),
A Philips medical systems Achieve scanner (3 T, 52 patients, 173 lesions), and
A Philips healthcare Ingenia scanner (3 T, 26 patients, 56 lesions)

Models were found by machine learning techniques, browsing recursively through the different variables and testing the performance including them one by one. A new variable was retained if the corresponding coefficient and the deviance it explained were statistically significant in the model and if its correlation to each of the other variables in the model was lower than 50%.

The threshold values were determined using an intermediate database containing MR images of prostates containing suspicious lesions, the benign or malignant nature of which had been established by biopsies, and chosen so as to obtain a sensitivity of 90%.

The intermediate database used MR data obtained from the same GE MR750 and Philips Healthcare Ingenia scanners as the learning database, with data from 101 patients and 11 patients, respectively.

The equations 1 to 3 and 5 were constructed from the selected parameters and their performance was subsequently evaluated on a test database comprising data (MRI images and biopsies) from different patients than the learning and intermediate database. While the learning database comprised only data from patients having confirmed prostate cancer, the test database comprised patients suspicious for prostate cancer, but for which cancer had not been confirmed.

The method for helping diagnosing prostate cancer was implemented on the MRI images of the test database, having two radiologists outline the first and second portions on the MRI images.

A total of 238 suspicious lesions were identified by radiologists on data from 158 patients, with 126 benign lesions (114 in the PZ, 12 in the TZ), 34 cancers having a Gleason score of 6 (30 in the PZ, 4 in the TZ) and 78 cancers having a Gleason score of 7 or more (71 in the PZ, 7 in the TZ), from biopsies.

The test database's MR images were acquired by four different MR scanners:
A General Electric Medical Systems Discovery MR 750 scanner (3 T, 62 patients, 93 lesions),
A Philips healthcare Ingenia scanner (3 T, 22 patients, 41 lesions),
A GE medical systems Optima MR450w (1.5 T, 66 patients, 91 lesions), and
A Philips Healthcare Ingenia (1.5 T, 8 patients, 13 lesions).

The sensitivity and specificity of systems using each one of equations 1, 2, 3 and 5 were estimated by comparison with diagnosis obtained from biopsies of the corresponding portions of the patient's prostates. The performance of systems was measured from the specificity (called “specificity sp90”) that was achieved when sensitivity was set to 90%.

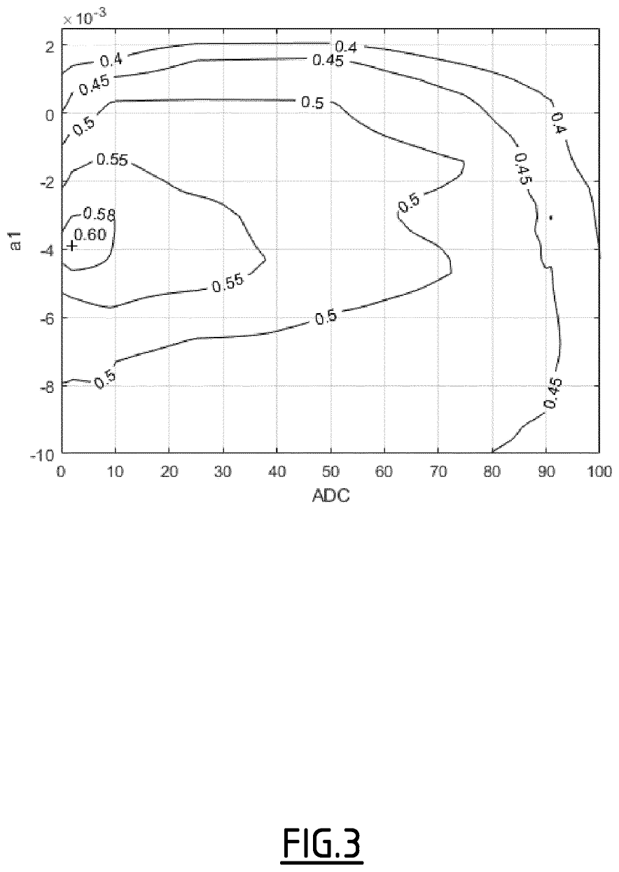

The accuracy of systems 10 using equation 1, with W being a normalized wash-in rate, consistently showed specificities above 0.5 when used on the test database, with ADC being a percentile inferior or equal to 40 and the first constant a₁ comprised between −6×10⁻³ and −10⁻³ mm². The values, for a system 10 using equation 1 in the test database, with W being a normalized wash-in rate, of specificity sp90 as a function of ADC and a₁ are shown on FIG. 3.

On FIGS. 3 to 5, the contours delimiting areas of specificity sp90 higher than threshold values are shown, for example the line marked “0.55” contains all areas of the data corresponding to sp90 higher than 0.55.

In particular, when ADC is a percentile inferior or equal to 30 and the first constant a₁ comprised between −5×10⁻³ and −3×10⁻³ mm², specificity was systematically over 0.55. When ADC is a percentile inferior or equal to 10 and the first constant a₁ comprised between −4.5×10⁻³ and −3×10⁻³ mm², specificity was substantially equal or superior to 0.58.

The best performances were obtained, on the test database, for ADC being the second percentile and the first constant a₁ being equal to −3.96×10⁻⁵ mm², with a specificity equal to 0.606.

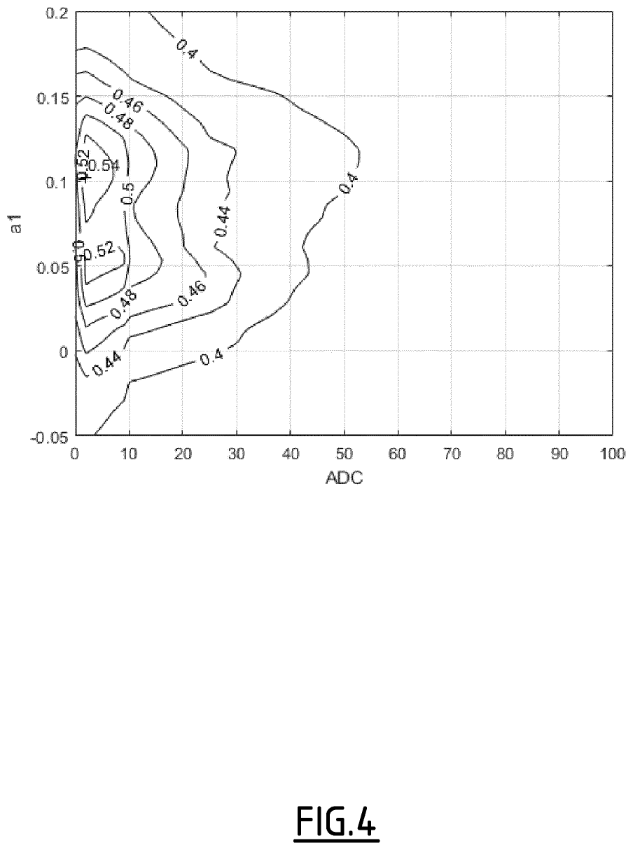

The accuracy of systems 10 using equation 1, with W being a normalized wash-out rate, consistently showed specificities above 0.45 when used on the learning database, with ADC being a percentile inferior or equal to 15 and the second constant a₁ comprised between 2×10⁻² and 15×10⁻² mm². The values, for a system 10 using equation 1, in the learning database, with W being a normalized wash-out rate, of specificity sp90 as a function of ADC and a₁ are shown on FIG. 4.

In particular, when ADC is a percentile comprised between 2 and 10 and the first constant a₁ comprised between 4×10⁻² and 12×10⁻² mm², specificity was systematically over 0.50.

The best performances were obtained, on the learning database, for ADC being the second percentile and the first constant a₁ being equal to 10×10⁻² mm², with a specificity equal to 0.549.

The accuracy of systems 10 using equation 2, consistently showed specificities above 0.45 when used on the test database, with ADC being a percentile comprised between 20 and 45 and the second constant b₁ is comprised between 10⁻⁷ and 14×10⁻⁷ mm².

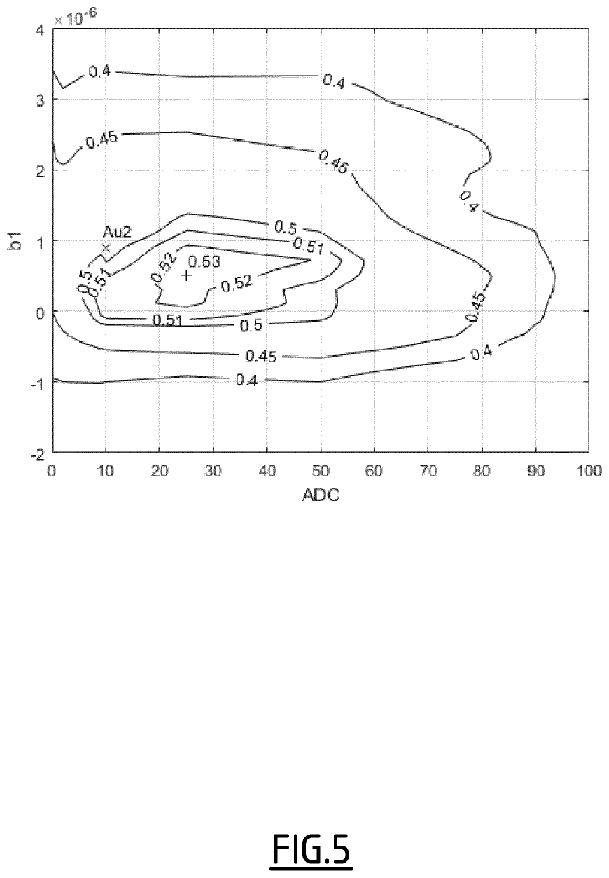

The values, for a system 10 using equation 2, in test learning database, of specificity sp90 as a function of ADC and b₁ are shown on FIG. 5.

In particular, when ADC is a percentile comprised between 20 and 45 and the second constant b₁ is comprised between 107 and 9×107 mm², specificity was systematically over 0.50.

The best performances were obtained, on the test database, for ADC being the twenty-fifth percentile and the second constant b₁ being equal to 5.1×10⁻⁷ mm², with a specificity equal to 0.539.

Examples of first threshold values and of the constants c₀ and c₁ determined are listed below, in different cases wherein the system 10 uses equation 4, with the quantity x being the third quantity x₃:
a) When ADC is the minimal percentile, the first threshold was comprised between 0.18 and 0.37, the seventh constant c₀ was comprised between 2.41 and 3.61, the eight constant c₁ was comprised between −5880 and −4020 s/mm² (the combination giving the best results was: first threshold equal to 0.28, seventh constant c₀ equal to 2.9, eight constant c₁ equal to −4790 s/mm²);
b) When ADC is the second percentile, the first threshold was comprised between 0.17 and 0.39, the seventh constant c₀ was comprised between 3.55 and 4.92, the eight constant c₁ was comprised between −7150 and −5270 s/mm² (the combination giving the best results was: first threshold equal to 0.27, seventh constant c₀ equal to 4.17, eight constant c₁ equal to −6080 s/mm²);
c) When ADC is the ninth percentile, the first threshold was comprised between 0.17 and 0.38, the seventh constant c₀ was comprised between 4.42 and 5.92, the eight constant c₁ was comprised between −7570 and −5660 s/mm² (the combination giving the best results was: first threshold equal to 0.27, seventh constant c₀ equal to 5.09, eight constant c₁ equal to −6500 s/mm²);
d) When ADC is the tenth percentile, the first threshold was comprised between 0.17 and 0.38, the seventh constant c₀ was comprised between 4.47 and 5.96, the eight constant c₁ was comprised between −7560 and −5650 s/mm² (the combination giving the best results was: first threshold equal to 0.26, seventh constant c₀ equal to 5.13, eight constant c₁ equal to −6490 s/mm²);
e) When ADC is the 25th percentile, the first threshold was comprised between 0.14 and 0.36, the seventh constant c₀ was comprised between 4.59 and 6.44, the eight constant c₁ was comprised between −7430 and −5260 s/mm² (the combination giving the best results was: first threshold equal to 0.27, seventh constant c₀ equal to 5.49, eight constant c₁ equal to −6260 s/mm²).

Examples of second threshold values and of the constants d₀ to d₁ determined are listed below, in different cases wherein the system 10 uses equation 6:
f) When ADC is the second percentile, the second threshold was comprised between 0.07 and 0.75, the ninth constant d₀ was comprised between 2.25 and 13.48, the tenth constant d₁ was comprised between −23820 and −4490 s/mm² (the combination giving the best results was: second threshold equal to 0.4, ninth constant d₀ equal to 6.5, tenth constant d₁ equal to −11520 s/mm²);
g) When ADC is the ninth percentile, the second threshold was comprised between 0.07 and 0.71, the ninth constant d₀ was comprised between 3.83 and 12.5, the tenth constant d₁ was comprised between −19500 and −6280 s/mm² (the combination giving the best results was: second threshold equal to 0.38, ninth constant d₀ equal to 7.3, tenth constant d₁ equal to −11320 s/mm²);
h) When ADC is the tenth percentile, the second threshold was comprised between 0.06 and 0.71, the ninth constant d₀ was comprised between 3.96 and 12.51, the tenth constant d₁ was comprised between −19100 and −6430 s/mm² (the combination giving the best results was: second threshold equal to 0.39, ninth constant d₀ equal to 7.4, tenth constant d₁ equal to −11360 s/mm²);
i) When ADC is the 25th percentile, the second threshold was comprised between 0.14 and 0.64, the ninth constant d₀ was comprised between 4.9 and 11.19, the tenth constant d₁ was comprised between −15030 and −6910 s/mm² (the combination giving the best results was: second threshold equal to 0.4, ninth constant d₀ equal to 7.9, tenth constant d₁ equal to −10740 s/mm²).

Systems 10 using equation 4, with the quantity x being the first quantity x₁, ADC being the second percentile, were found to have the following AUC and specificity when using the outlines of one of the radiologists, (the sensitivity and specificity % when using the outlines of the other radiologist being indicated in parentheses), in the following cases:
Case a: AUC between 76% and 86% (between 78 and 87%), specificity sp90 33 to 52% (28 to 63%),
Case b: AUC between 77% and 86% (between 79 and 87%), specificity sp90 34 to 54% (29 to 66%),
Case c: AUC between 78% and 88% (between 79 and 88%), specificity sp90 37 to 61% (32 to 74%),
Case d: AUC between 79% and 88% (between 79 and 88%), specificity sp90 37 to 62% (32 to 74%),
Case e: AUC between 79% and 88% (between 80 and 89%), specificity sp90 39 to 62% (38 to 72%).

“AUC” is the performance as estimated from the area under a curve of the true positive rate as a function of the false positive rate. Such curves are well-known in the art and often called “Receiver Operating Characteristic (ROC) curves”.

Systems 10 using equation 5 were found to have the following AUC and specificity when using the outlines of one of the radiologists, (the sensitivity and specificity % when using the outlines of the other radiologist being indicated in parentheses), in the following cases:
Case f: AUC between 47% and 86% (between 35 and 83%), specificity sp90 0 to 80% (0 to 82%),
Case g: AUC between 46% and 88% (between 28 and 85%), specificity sp90 0 to 83% (0 to 82%),
Case h: AUC between 46% and 89% (between 30 and 85%), specificity sp90 0 to 83% (0 to 82%), and
Case i: AUC between 50% and 94% (between 29 and 82%), specificity sp90 0 to 83% (0 to 75%).

It should be noted that both quantities x₁ and x₂ combine a centile of ADC and of a dynamic quantity of the MRI images (i.e a quantity that reflects changes as a function of time of a value during inflow or outflow of the contrast agent in or out of the prostate), i.e a wash-in rate, a wash-out rate or a time-to-peak value.

Therefore, part of the invention lies in the discovery, by the inventors, that such combinations are reflective of a likelihood that a cancer is present in a portion of the prostate, and in the discovery of ranges of values a₁, b₁ and of ADC centiles that, when combined together, lead to an efficient discovery of cancers from the images.

The computer aided diagnosis method described herein may be used to establish a clinical diagnosis based on the results of said method for helping diagnosing aggressive prostate cancers.

In some embodiments, the methods of the present invention are performed in vitro or ex vivo.

Additional validation studies have been performed by the inventors to confirm the initial results of the method. These results are provided below solely as an example.

In this example, the evaluation was performed on another test dataset (named ‘external test dataset’) independent from the test dataset described above.

This new database is composed of 104 patients with 126 lesions imaged on one scanner, a GE medical systems Signa Voyager scanner (1.5 T, 104 patients, 126 lesions).

These 104 patients have suspected cancer and underwent mpMRI and subsequent biopsy in another institution.

As in the previously described test, the PI-RADSv2 scores (Prostate Imaging-Reporting and Data System version 2) prospectively assigned to each lesion at the time of biopsy were retrieved from the patients' medical records. Then, based on mpMRI reports, two radiologists, with 19 (OR, R1) and 5 (PCM, R2) years of experience, blinded to each other and to biopsy findings, retrospectively outlined the targeted lesions on T2-weighted, diffusion-weighted and DCE images, on the slice level they considered the most representative. The calculation of the lesions' score was performed using the PZ or the TZ model, depending on their location. Thus, each lesion received a PI-RADSv2 score (prospectively assigned at the time of biopsy) and two CAD scores using the regions of interest (ROI) delineated by R1 and R2. The PI-RADSv2 scores and the scores obtained through the invention were then compared to biopsy findings.

The table below details the distribution of the PI-RADSv2 scores and the invention-derived scores as a function of biopsy results.

| | PI- | | Biopsy findings | | | | |
|---|---|---|---|---|---|---|---|
| | RADSv2 | CAD | | ISUP | ISUP | ISUP | |
| | score | result | Benign | 1 | 2 | ≥3 | Total |
| ROI | 1 | CAD− | 8 | 0 | 0 | 0 | 8 |
| delinea- | | CAD+ | 0 | 0 | 0 | 0 | 0 |
| tions by | 2 | CAD− | 3 | 1 | 0 | 0 | 4 |
| practi- | | CAD+ | 0 | 0 | 0 | 0 | 0 |
| tioner | 3 | CAD− | 8 | 1 | 0 | 0 | 9 |
| R1 | | CAD+ | 1 | 3 | 0 | 0 | 4 |
| | 4 | CAD− | 18 | 9 | 0 | 2 | 29 |
| | | CAD+ | 2 | 12 | 6 | 1 | 21 |
| | 5 | CAD− | 3 | 0 | 0 | 1 | 4 |
| | | CAD+ | 1 | 3 | 8 | 13 | 25 |
| | Total | | 44 | 29 | 14 | 17 | 104 |
| ROI | 1 | CAD− | 8 | 0 | 0 | 0 | 8 |
| delinea- | | CAD+ | 0 | 0 | 0 | 0 | 0 |
| tions by | 2 | CAD− | 3 | 1 | 0 | 0 | 4 |
| practi- | | CAD+ | 0 | 0 | 0 | 0 | 0 |
| tioner | 3 | CAD− | 9 | 3 | 0 | 0 | 12 |
| R2 | | CAD+ | 0 | 1 | 0 | 0 | 1 |
| | 4 | CAD− | 18 | 10 | 0 | 2 | 30 |
| | | CAD+ | 2 | 11 | 6 | 1 | 20 |
| | 5 | CAD− | 4 | 1 | 0 | 1 | 6 |
| | | CAD+ | 0 | 2 | 8 | 13 | 23 |
| | Total | | 44 | 29 | 14 | 17 | 104 |

In the table above, data indicate numbers of patients. ISUP is the abbreviation of the International Society of Urological Pathology grade measuring the severity of a cancer. CAD is the abbreviation of the computer-aided diagnosis system according to the invention.

In the new test dataset, the score AUCs were 82% (95% Cl: 76-89) and 86% (95% Cl: 79-93) using the ROIs delineated by R1 and R2 respectively. They were not significantly different from that of the PI-RADSv2 score assigned at the time of biopsy (85%, 95% Cl: 79-91, p=0.82 and p=1 respectively).

This new test was challenging since all the patients were imaged a scanner different and more recent than those used in the training dataset. Despite this, the system according to the invention provided robust results, not only in terms of overall diagnostic performance (as quantified by the AUC), but also in terms of diagnostic thresholds (as quantified by sensitivity and specificity). Remarkably, the thresholds that provided a 90% sensitivity in the pre-test dataset provided similar sensitivities in the internal (85-89%) and external (92%) test datasets. At this level of sensitivity, the specificity of the computer-aided diagnosis system according to the invention was good (64-76%) suggesting that it could offer a better sensitivity/specificity trade-off than the PI-RADSv2 classical thresholds.

1. A system for helping diagnosing aggressive prostate cancers, the system comprising a control module, the control module being configured to:
receive at least one magnetic resonance image of at least a first portion of the peripheral zone of a subject's prostate, the image comprising a set of pixels, the image comprising, for each pixel, an apparent diffusion coefficient value of a corresponding area of the prostate,
calculate a first score, and
determine that the first portion contains an aggressive cancer if a first criterion based on the calculated first score is verified, or that the first portion does not contain an aggressive cancer if the first criterion is not verified, and
the first score being calculated as a function of at least one of the following quantities x₁ and x₂:
x₁=ADC+a₁×W, wherein W is a value of a normalized wash-in rate or a normalized wash-out rate of the first portion, the wash-in or wash-out value being expressed in Hertz and being estimated from a set of magnetic resonance images of the first portion, ADC being a percentile of the apparent diffusion coefficient values of the first portion and being expressed in units of mm²/s, a₁ being a first constant, the first constant being comprised between −6×10⁻³ and −10⁻³ mm² and the percentile being inferior or equal to 40 when W is a normalized wash-in rate value, the first constant being comprised between 2×10⁻² and 15×10⁻² mm² and the percentile being inferior or equal to 15 when W is a normalized wash-out rate value,
x₂=ADC+b₁×TTP, wherein b₁ is a second constant comprised between 10⁻⁷ and 14×10⁻⁷ mm²/s², ADC is a percentile comprised between 20 and 45 of the apparent diffusion coefficient values of all pixels corresponding to the first portion, and TTP is a time-to-peak value of the first portion, expressed in seconds, the time-to-peak value being a time duration from a first instant to a second moment, the first instant being a time of arrival of a contrast agent in the prostate, the second instant being a time of maximum contrast concentration of an MRI signal of the first portion.

2. The system according to claim 1, wherein W is a normalized wash-in rate and the first constant a₁ is comprised between −5×10⁻³ and −3×10⁻³ mm².

3. The system according to claim 2, wherein the percentile is comprised between 0 and 30.

4. The system according to claim 1, wherein the second constant b₁ is comprised between 10⁻⁷ and 9×10⁻⁷ mm²/square second.

5. The system according to claim 1, wherein ADC is the twenty-fifth percentile of the apparent diffusion coefficient values of all pixels corresponding to the first portion when the first score is calculated as a function of the time-to-peak value.

6. The system according to claim 1, wherein a wash-in or wash-out value is estimated for each pixel corresponding to the first portion, W being a mean value of the wash-in or wash-out values estimated for the pixels.

7. The system according to claim 1, wherein a plurality of images of the first portion are acquired during a time period, the time period being a time period wherein a contrast agent enters the prostate or a time period wherein a contrast agent leaves the prostate, each image comprising, for each pixel, an intensity value of a T1-weighed signal of the corresponding area of the prostate, the control module being configured to calculate, for each image, a mean value of normalized intensity values of the pixels corresponding to the first portion, each normalized value being equal to the measured value divided by the value of the same pixel before the contrast agent enters the prostate, the normalized wash-in rate value being an ascending slope of a curve of the calculated mean values as a function of time, and the normalized wash-out rate being a descending slope of the curve.

8. The system according to claim 1, wherein the control module is configured to compare the score with a threshold and to determine that the first portion comprises an aggressive cancer if the first quantity is inferior or equal to the threshold.

9. The system according to claim 8, wherein the score is equal to the first quantity x₁, the threshold being comprised between −0.8×10⁻³ and 2.3×10⁻³ square millimetres per second when W is a normalized wash-in rate or the threshold being comprised between −1.1×10⁻³ and 3.2×10⁻³ square millimetres per second when W is a normalized wash-out rate.

10. The system according to claim 8, wherein the score is equal to the second quantity x₂, the threshold being comprised between −1.1×10⁻³ and 3.3×10⁻³ square millimetres per second.

11. The system according to claim 1, wherein each image comprises an image of a second portion of the transition zone of the prostate, the control module being further configured to:
calculate a second score Y as a function of a fourth quantity y, the fourth quantity y being according to the equation:
y=d₀+d₁×ADC
wherein d₀ is a fifth constant, d₁ is a sixth constant comprised between −23820 and −4490 seconds/mm², the fifth constant being notably comprised between 2.25 and 13.48, and ADC is a percentile inferior or equal to 25 of the apparent diffusion coefficient values of all pixels corresponding to the second portion, and
determine that the second portion comprises an aggressive cancer if a second criterion based on the calculated second score Y is verified, or that the second portion does not contain an aggressive cancer if the first criterion is not verified.

12. A method for helping diagnosing aggressive prostate cancers, the method being implemented by a system comprising a control module, the method being implemented by the control module and comprising steps for:
receiving at least one magnetic resonance image of a first portion of the peripheral zone of a subject's prostate, the image comprising a set of pixels, the image comprising, for each pixel, an apparent diffusion coefficient value of a corresponding area of the first portion,
calculating a first score, and
determining that the first portion comprises an aggressive cancer based on the calculated first score,
the first score being calculated by the control module as a function of at least one of the following quantities x₁ and x₂:
x₁=ADC+a₁×W, wherein W is a value of a normalized wash-in rate or a normalized wash-out rate of the first portion, the wash-in or wash-out value being expressed in Hertz and being estimated from a set of magnetic resonance images of the first portion, ADC being a percentile of the apparent diffusion coefficient values of the first portion and being expressed in units of mm²/s, a₁ being a first constant, the first constant being comprised between −6×10⁻³ and −10⁻³ mm² and the percentile being inferior or equal to 40 when W is a normalized wash-in rate value, the first constant being comprised between 2×10⁻² and 15×10⁻² mm² and the percentile being inferior or equal to 15 when W is a normalized wash-out rate value,
x₂=ADC+b₁×TTP, wherein b₁ is a second constant comprised between 10⁻⁷ and 14×10⁻⁷ mm²/s², ADC is a percentile comprised between 20 and 45 of the apparent diffusion coefficient values of all pixels corresponding to the first portion, and TTP is a time-to-peak value of the first portion, expressed in seconds, the time-to-peak value being a time duration from a first instant to a second moment, the first instant being a time of arrival of a contrast agent in the prostate, the second instant being a time of maximum contrast concentration of an MRI signal of the first portion.

13. A computer program product containing software instructions configured to implement a method according to claim 12 when the software instructions are executed on a processor.

14. An information medium containing software instructions configured to implement a method according to claim 12 when the software instructions are executed on a processor.

15. A diagnosis method, comprising: using the method for helping diagnosing aggressive prostate cancers according to claim 12, and establishing a clinical diagnosis based on the results of said method for helping diagnosing aggressive prostate cancers.
